(19) United States
(12) Patent Application Publication
(10) Pub. No.: US 2015/0218662 A1
(43) Pub. Date: Aug. 6, 2015

(54) METHOD FOR DETECTING AND TYPING NUCLEIC ACIDS OF PATHOGENIC MICROORGANISM WITHOUT AMPLIFICATION

(71) Applicant: SOUTHWEST HOSPITAL OF CHONGQUING, Chongging (CN)

(72) Inventors: Yang Luo, Shaanxi (CN); Bo Zhang, Chongqing (CN); Tianlun Jiang, Chongqing (CN); Weiling Fu, Chongqing (CN); Wei Liu, Chongqing (CN)

(21) Appl. No.: 14/423,638

(22) PCT Filed: Jun. 28, 2013
(86) PCT No.: PCT/CN2013/000781

§ 371 Date: Feb. 24, 2015

(30) Foreign Application Priority Data
Aug. 30, 2012 (CN) 201210326601.2

(51) Int. Cl. C12Q 1/70

(52) U.S. Cl. CPC C12Q 1/706; C12Q 2600/158

(57) ABSTRACT

The invention discloses a method for directly detecting and typing nucleic acids of pathogenic microorganism without amplification and a related kit, the invention achieves detecting and typing nucleic acids of pathogenic microorganism without amplification by the combination of multiprobe and the layer by layer assembly of fluorescence quantum dots. The method according to the invention can directly detect a nucleic acid with low concentration without amplification; the multiprobe prevents the false positives which are likely to occur in the process of signal amplification and thus increases detection accuracy. Such technology can achieve the real-time detection and simultaneous genotyping of pathogenic microorganisms with rapid speed and low cost.

### TECHNICAL FIELD

The invention is directed to biological medicine, particularly to a method and a kit for directly detecting and typing nucleic acids of pathogenic microorganism without amplification.

### BACKGROUND ART

Infectious diseases are one of the most important diseases that greatly threaten human health. According to the statistics from Centers for Disease Control (CDC), there are 6,320,000 cases of notifiable diseases with a death of 15,000 in our country in 2011. Among them, viral hepatitis, pulmonary tuberculosis and syphilis rank among the top three in terms of morbidity, and the three diseases account for 85.41% of the total morbidity of category B infectious diseases. The morbidity of hematogenic infectious diseases such as hepatitis B, hepatitis C increases year by year. As shown in the above data, hepatitis B still ranks first in morbidity of infectious diseases in our country, and the number of cases thereof accounts for more than 70% of all hepatitis cases in our country. Various clinical data and research has illustrated that the serologic outcome and prognosis of a patient with hepatitis B is closely associated with the genotype and copy number of the infected hepatitis B virus (HBV). Therefore, it is of great clinical significance to establish a rapid, accurate HBV detection and typing method for the early diagnosis, efficacy monitoring, prognosis judgment and individual therapy.

For the detection of HBV infection, the present laboratory methods are mainly divided into two classes: direct detection and indirect detection. The indirect detection is mainly based on biochemistry methods and immunology methods. The biochemistry methods indirectly determine viral infection by detecting the raise of several transaminases (ALT, AST, γ-GGT etc.), their sensitivity is relative higher but they are easily affected by liver injuries caused by other factors and thus the specificity thereof is poor. The immunological methods include early ELISA and gradually formed immune nephelometry, chemiluminescence and time-resolved fluorescence etc. The principle thereof is comprehensive judgment by measuring several HBV special antigens (HBsAg, HBcAg, HBeAg) and corresponding antibodies (HBsAb, HBcAb) produced in the body of a patient. Such methods are easily performed and wildly used in clinic. However, immunological methods cannot detect the HBV infection during “window phase” and readily lead to false negatives. Importantly, all the indirect detection methods cannot perform HBV genotyping and thus cannot direct the individual clinical drug use.

The direct detection methods detect the number and genotype of HBV in samples from patients, which is characterized by early, real-time, dynamic monitoring of the copy number of HBV, and such methods have incomparable advantages in the aspects of early diagnosis, efficacy judgment and individual therapy. The viral direct detection is all achieved by detecting HBV nucleic acids at present for virus is extremely hard to culture in vitro. However, the copy number of HBV in the body of a patient with early HBV infection is lower (generally 10⁴ to 10⁶/ml), which is not adequate to be detected by conventional molecular biological methods such as nucleic acid hybridization. Therefore, the amplification of target molecule signal is the premise of the high resolution detection and typing of the HBV DNA. The main strategy of signal amplification comprises the amplification of DNA template (pre-amplification) and the amplification of detection signal (post-amplification). The DNA template amplification technology is based on PCR to achieve signal amplification by amplifying the nucleic acid template to 10⁹ in vitro. A series of heterotherm nucleic acid amplification and detection technologies such as nested PCR, fluorescence quantitative PCR and multiple PCR are derived from PCR technology. Such PCR-based amplification technologies used in HBV detection and typing have the following disadvantages: (1) the amplification is quite strict and false positives or false negatives are easily produced; (2) simultaneous amplification of various genotypes often leads to the competitive inhibition of a template of a low concentration with the template of a high concentration, which results in the false negative of the template of a low concentration; (3) the barrier of the core intellectual properties relative to PCR leads to expensive reagents and instruments thereof, which increases the medical cost and burden of a patient. A series of isothermal amplification technologies have been developed in recent years, such as strand displacement amplification (SDA), loop-mediated isothermal amplification (LAMP), rolling circle amplification (RCA) etc., which partly reduce medical cost and solve the above problem of inhibition of the template of low concentration. However, these technologies still cannot perform high resolution detection and genotyping of HBV simultaneously.

With respect to DNA template amplification technology, the detection signal amplification technology (post-amplification) only amplifies a detected low signal and eliminates the amplification inhibition produced by amplification of templates with different concentration. As the detection signal amplification technology is closely related with detection principle, each detection technical platform has its most suitable signal amplification technology, such as mass amplification based on quartz crystal microbalance (QCM) sensor, refraction angle amplification based on surface plasmons (SPR) sensor, enzymatic amplification based on electrochemical sensor, fluorescence enhancement based on nanosensor for fluorescence detection, etc. Among these detection platforms, biological sensing technology is used to transfer a weak signal below the detection limit into a recognizable physical or chemical signal. The most used before is an enzymatic sensor, which amplifies a signal by enzymatic catalysis or binding to the substrate. In recent years, the fast development of nano-material synthesis and surface modification technology has provided wide space for the research and development of signal amplification technologies. The inventor has made considerable research on nano-material signal amplification and successfully applied gold nano-particles to the signal amplification in a QCM sensor, and thus achieved detection of Staphylococcus aureus with a low concentration in blood and the amplification of a non-enzymatic fluorescence signal in HCR reaction. However, we have found that the traditional fluorescence dye is readily bleached in assays, which makes it hard to be detected in a clinical sample.

However, the sequence homology among the A-H subtypes of HBV is very high, so that a probe with extremely high specificity is necessary to prepared for typing of HBV with nucleic acid hybridization. Therefore, the present HBV typing technologies firstly classify each subtype and then several sets of DNA probes are used to detect sets of different genotypes respectively to increase detection specificity. Comparing to a DNA molecule, the affinity constant of binding between a peptide nucleic acid (PNA) molecule and a single DNA strand is 10³ time of that of normal DNA-DNA binding, therefore a short strand PNA probe (14-20 bp) has strong ability to recognize a single base mutation, the highly specific recognization ability of a PNA probe provides a new breakthrough for HBV virus genotyping.

### SUMMARY OF THE INVENTION

The technical problem to be solved in the invention is to provide a method for detecting and typing nucleic acids of pathogenic microorganism without amplification. In order to achieve the object of the invention, the following technical solutions are used:

The invention is directed to a method for detecting and typing nucleic acids of pathogenic microorganism without amplification, wherein the method comprises the following steps:

(1) Synthesizing DNA and/or PNA probe 1, 2, 3 according to the nucleic acid sequence of a sample to be tested, the probe 1, 2, 3 can hybridize to the sample to be tested respectively without overlapping with each other;

(2) Coupling the probe 1, 2, 3 with a magnetic nanoparticle and two fluorescence quantum dots respectively, the fluorescence of the fluorescence quantum dots can be same or different;

(3) Synthesizing biotin-linked bridged DNA and/or PNA sequences 1, 2 and complementary sequences 1′, 2′, the sequences 1′ and 2′ are coupled with the two fluorescence quantum dots in step (2) respectively;

(4) Synthesizing two biotin-modified fluorescence quantum dots with different fluorescence colors, the two fluorescence quantum dots and the fluorescence quantum dots in step (2) can be same or different;

(5) Selecting the probe-modified magnetic nanoparticle and one of the probe-modified fluorescence quantum dots in step (2), which hybridize to the sample to be tested and corresponding bridged sequences, followed by magnetic separation; then layer by layer assembly is performed by repeating as follows: adding Sa (streptavidin)-wash-adding one of the biotin-modified fluorescence quantum dots in step (4)-wash, then the concentrate of the sample to be tested is obtained by magnetic separation, optionally a sample of the concentrate is measured for fluorescence intensity;

(6) Selecting the other probe-modified fluorescence quantum dot, which then hybridizes to the concentrate from step (5) and the corresponding bridged sequence, followed by magnetic separation; then layer by layer assembly is performed by repeating as follows: adding Sa-wash-adding one of the biotin-modified fluorescence quantum dots in step (4)-wash, then the second concentrate of the sample to be tested is obtained by magnetic separation, optionally a sample of the second concentrate is measured by fluorescence spectral imaging technology or flow cytometry.

Another aspect of the invention is directed to a kit for directly detecting and typing nucleic acids of pathogenic microorganism without amplification, wherein the kit comprises: three DNA and/or PNA probe-coupled magnetic nanoparticles and two fluorescence quantum dots, the three DNA and/or PNA probe-coupled magnetic nanoparticles can hybridize to a sample to be tested without overlapping with each other, the fluorescence of the fluorescence quantum dots can be same or different; biotin-modified bridged DNA and/or PNA, two fluorescence quantum dots coupled with the complementary sequence of bridged DNA and/or RNA; two biotin-modified fluorescence quantum dots with different fluorescence colors, and the fluorescence of the two fluorescence quantum dots and the above fluorescence quantum dots can be same or different; SA and a buffer.

In a preferred embodiment according to the invention, wherein the sample to be tested is a HBV nucleic acid.

In a preferred embodiment according to the invention, the probe is PNA, one or more or all of the fluorescence quantum dots are CdSe/ZnS quantum dot.

In a preferred embodiment according to the invention, the magnetic nanoparticle is SiO₂@Fe₃O₄ nanoparticle.

In a preferred embodiment according to the invention, said three probes are PNA, wherein two species-specific sequences have the sequence of probe 1 or probe 2 in the following table, said biotin-modified bridged DNA sequence is shown in the following table.

| Probe name | Probe sequence |
|---|---|
| PNA species-specific | 5′-NH₂-(CH2)₆-AGGCACAGCTTGG |
| probe 1 | AGGC-3′ |
| PNA species-specific | 5′-NH₂-(CH2)₆-GTGATGTGCTGGG |
| probe 2 | TGTGTCG-3′ |
| Bridged DNA sequence | 5′-biotin-GGGCAGCTGGGGCGGG |
| | CGGG-NH₂-3′ |

The other sequence for typing is selected from one of the following three probes:

| Probe name | Sequence |
|---|---|
| P3b | 5′-NH₂-(CH2)₆-TGTGTTTACTGAGTG-3′ |
| P3c | 5′-NH₂-(CH2)₆-AACGCCCACATGATCT-3′ |
| P3d | 5′-NH₂-(CH2)₆-CGGTACGAGATCTTCTA |

In a preferred embodiment according to the invention, the method is not for diagnose.

The method according to the invention can be used to directly detect a nucleic acid with low concentration without amplification; multiprobe prevents the false positives which are likely to appear in the process of signal amplification and thus increases detection accuracy. Such technology can achieve the real-time detection and simultaneous genotyping of pathogenic microorganisms with rapid speed and low cost.

### DESCRIPTION OF FIGURES

FIG. 1 schematically shows the detection principle;

FIG. 2 shows the SEM graph of the synthesized CdSe/ZnS quantum dot;

FIG. 3 shows the SEM graph of the synthesized super paramagnetic Fe₃O₄;

FIG. 4 shows the DLS graph of a quantum dot;

FIG. 5 shows the DLS graph of a magnetic microsphere;

FIG. 6 schematically shows the synthesis of a polymer containing a biotin ligand;

FIG. 7 schematically shows the synthesis and modification of a quantum dot;

FIG. 8 shows the electrophoresis of coupling a DNA probe of different molar ratio with a quantum dot;

FIG. 9 shows the fluorescence spectrum after coupling DNA probe of different mole ratio with a quantum dot;

FIG. 10 shows the relationship between different QD self-assembly layer numbers and the amplification of fluorescence signal;

FIG. 11 shows the detection fluorescence spectrum result of HBV virus of different concentrations;

FIG. 12 shows the stand curve of HBV detection of different concentrations;

FIG. 13 shows the comparison of detection results of different mismatched sequence hybridization (specificity);

FIG. 14 shows the detection results of detection and simultaneous typing (540 nmQD for detection, 620 nmQD for typing).

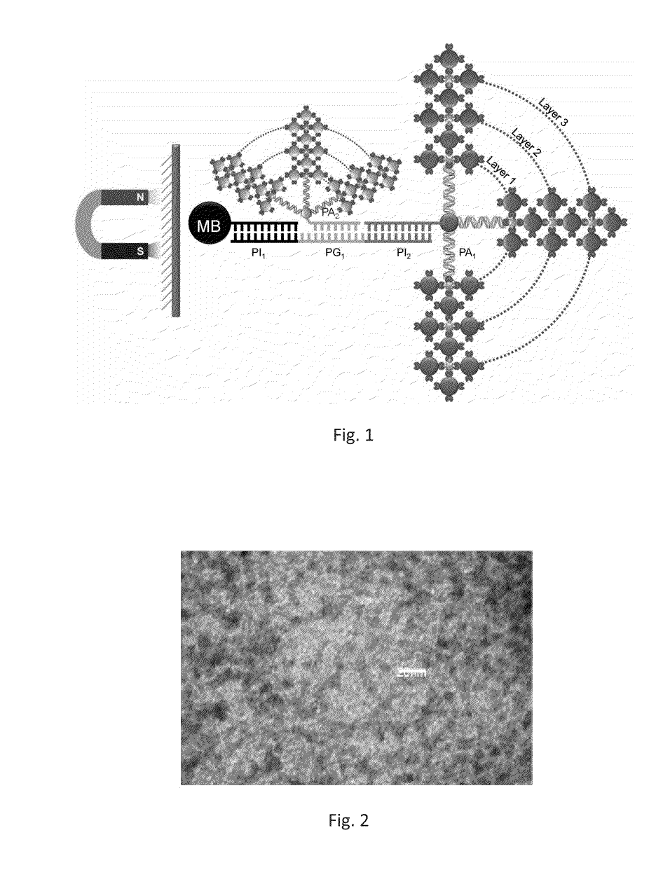

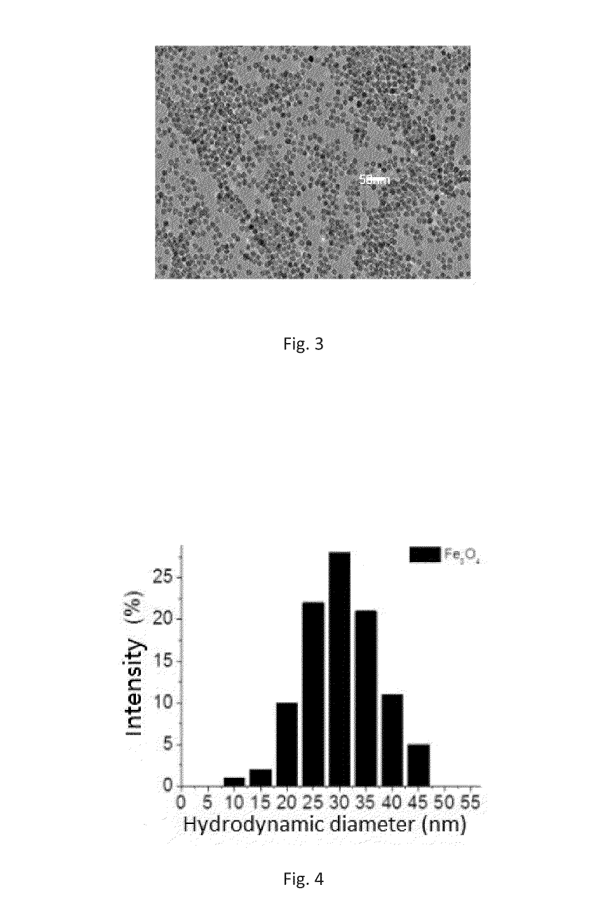

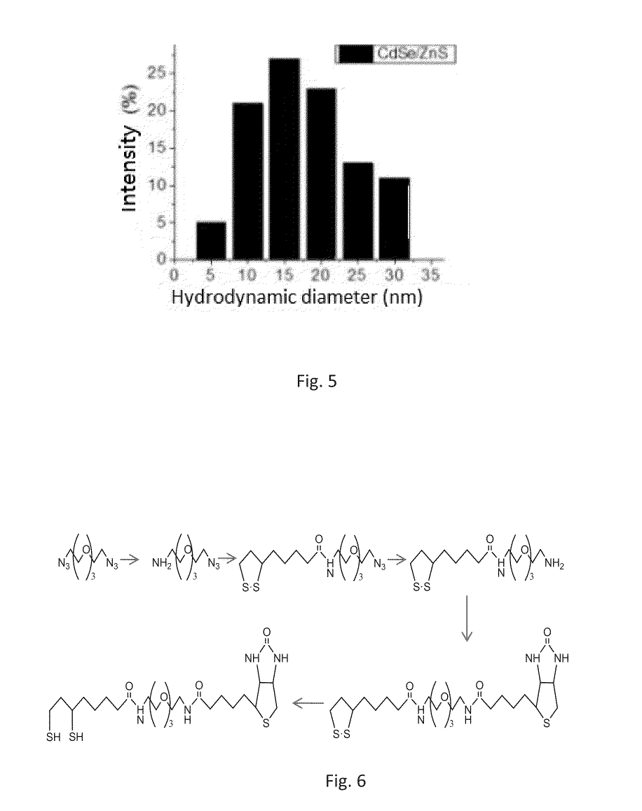

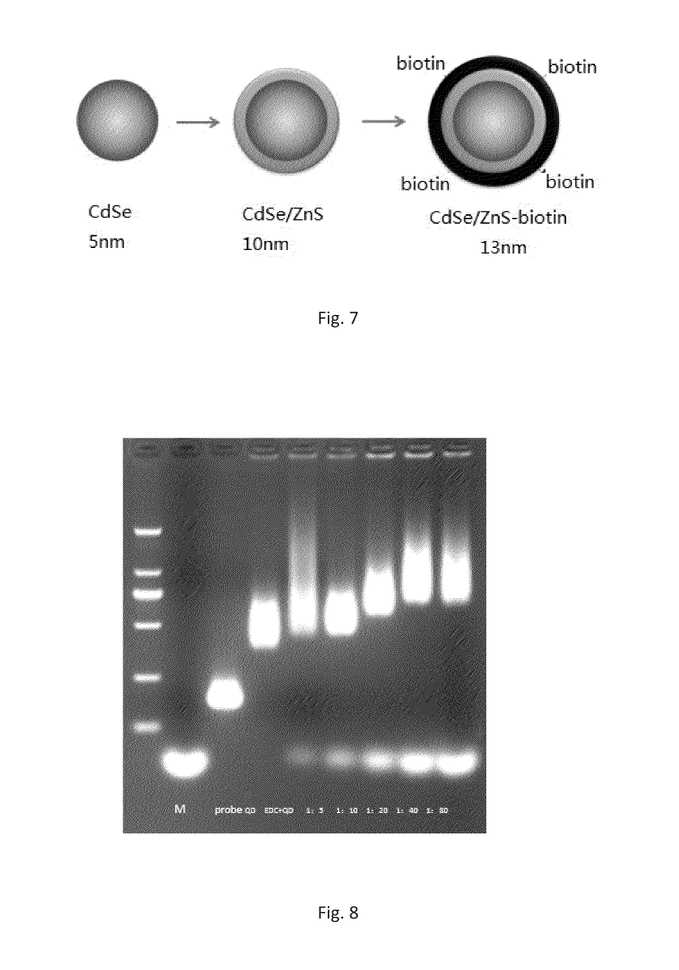

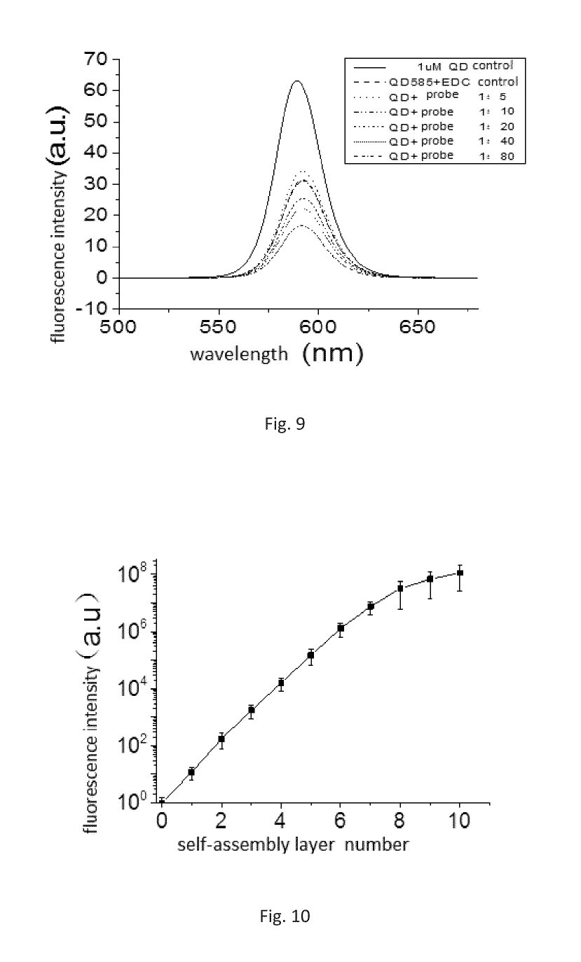

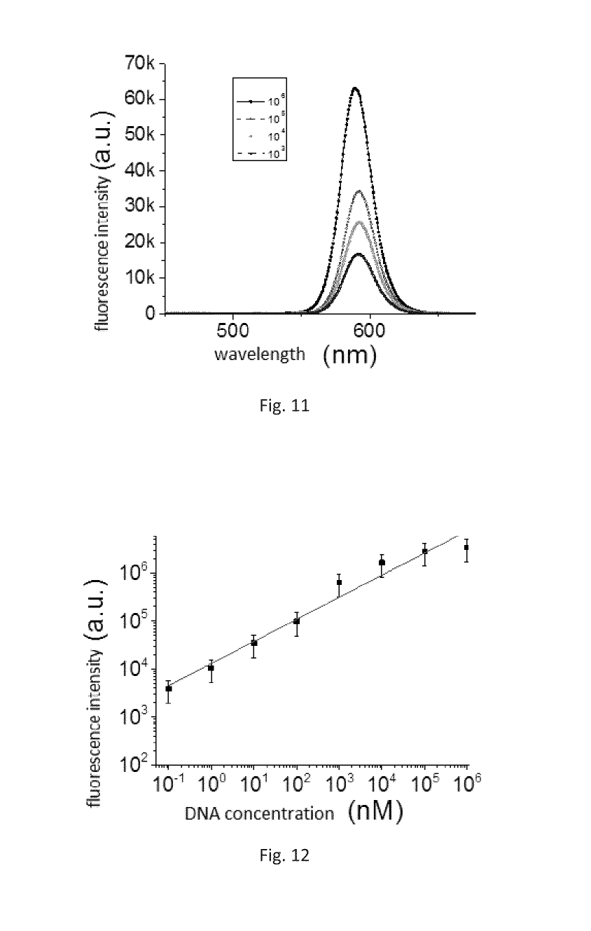

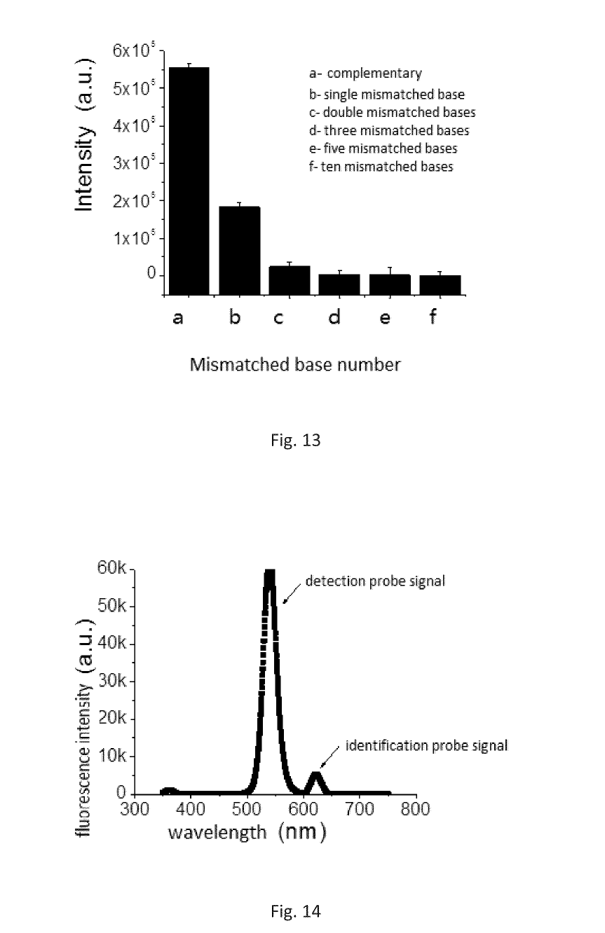

### DETAILED DESCRIPTION OF THE INVENTION

(1). Preparation of HBV Identification Probe

For DNA probe, HBV probes are designed with the combination of oligo 6.0 software and primer Premier 6.0 software. With respect to the design of PNA probe, after several candidate sequence regions are searched with the above software (the candidate sequence regions are expended to more than one time), several candidate sequences are searched with oligonucleotide software (at ratio of 1:10), then the candidate sequences are filed to a PNA synthesis company (Bio-Synthesis) for sequence verification and finally the synthesized PNA probe has a length of 14 to 20 bp. The verification and synthesis of PNA probe is both accomplished by Bio-Synthesis. The design principle of bridged DNA probe is to achieve high Tm without a loop structure based on the premise that the sequence is short.

| Probe name | Probe sequence |
|---|---|
| PNA species-specific | 5′-NH₂-(CH₂)₆-AGGCACAGCTTGGA |
| probe 1 | GGC-3′ |
| PNA species-specific | 5′-NH₂-(CH₂)₆-GTGATGTGCTGGGT |
| probe 2 | GTGTCG-3′ |
| Bridged DNA sequence | 5′-biotin-GGGCAGCTGGGGCGGGC |
| | GGG-NH₂-3′ |

(2). Synthesis and Characterization of Multicolour Quantum Dot Nanoparticle

Synthesis of CdSe/ZnS quantum dot: 156 mg NaBH₄ is dissolved in 2 mL of water under an oxygen-free condition. 157.8 mg Se powder is added after ultrasonic mixing and colorless NaHSe solution is produced. The reaction equation is: 4NaBH₄+2Se+7H₂O=2NaHSe+Ha₂B₄O—↓+14H₂↑.

228.5 mg CdCl₂.25H₂O is accurately weighed and dissolved in 100 ml of distilled water, and the solution is poured into a conical flask. After pumping nitrogen for 30 minutes, 262 μl of 3-hydroxypropionic acid is added dropwisely, and then the pH is adjusted to 11.0 with NaOH. Nitrogen is continuously pumped into a flask for 30 to 40 minutes to remove O₂, and 1 ml of prepared NaHSe solution is slowly added into the flask at the same time. The reaction container is sealed after vigorous stirring with a magnetic stirrer, the solution of CdSe quantum dot is obtained after refluxing in water bath at 95° C. for 1 h. The prepared CdSe solution is cooled to room temperature and wherein nitrogen is pumped for 30 minutes under vigorous magnetic stirring, 88 mg Zn(Ac)₂.2H₂O and 96 mg Na₂S.9H₂O is slowly added to formulate a 10 mL solution, which is in water bath at 95° C. for 2 h to obtain the solution of CdSe quantum dot. According to the above processes, various quantum dots with different wavelength (proposed preliminarily 525 nm, 550 nm, 565 nm, 605 nm, 620 nm) can be prepared by controlling the reflux time.

Characterization of quantum dot: the fluorescence emission spectrum and visible absorption spectrum of CdSe/ZnS quantum dots with different emission wavelength is detected with fluorescence spectrophotometer and double beam UV visible spectrophotometer respectively. The nanoparticle size, particle size distribution and surface Zeta charge of nanoparticle dispersion is measured with laser light scattering instrument. The prepared nanoparticle dispersion is dropped on copper screen coated with carbon film, after drying at room temperature, the particle size distribution of QD nanoparticle is observed with transmission electron microscope. The electron diffraction diagram is used to determine the condition of the diffraction ring of CdSe/ZnS quantum dots. The reaction conditions of quantum dot synthesis (pH, mole ratio, reflux time, etc.) are optimized according to the above results.

Surface modification and characterization of quantum dot: the surface biotin modification of quantum dot is performed mainly according to the methods reported by HediMattoussi etc, and the principle thereof is to first synthesize a polymer with biotin-modified surface, the quantum dot packaged in the polymer has the advantage of small size and controllable coupling site of high liquid phase dispersion. The detailed method is as follows: first synthesizing (1) Diazide functional tetraglycol, purifying the synthesized product (1), then adding 250 ml of 0.7M phosphoric acid, and 110 mmol triphenylphosphine (PPh₃) to react for 16 h, obtaining monamine-modified tetraglycol after wash, filter, extraction and drying. Then 55.8 mmol lipoic acid, 10.3 mmol 4-dimethylamino CH₂Cl₂ is added followed by cooling to zero, then 53.8 mol DCC is slowly added to react for 16 h, TA-TEG-N3 complex is obtained by filter and column purification, and then 150 ml THF and 81 mmol PPh₃ is added to react for 20 h, amino end labeled TA-TEG is obtained after separation and purification, then hydroxylated biotin is added and reacted in DMF for 16 h, TA-TEG-biotin is obtained after separation and purification. 18.5 mmol NaBH₄ is added and reacted in 75% ethanol for 4 h, after extracting with chloroform and column purification, DHLA-TEG-biotin is obtained. Then CdSe/ZnS solution covered with TOP/TOPO is added and heated to 60 to 80° C. to react for 6 to 12 h. After precipitation with a mixture of n-hexane, ethanol and chloroform (11:10:1), it is dispersed in water. Finally the solution of biotin-modified CdSe/ZnS quantum dot (CdSe/ZnS-biotin) is obtained.

The diameter (10-20 nm) of formed microspheres is observed by TEM, SEM electron microscopy for surface-modified quantum dot, the hydration diameter in double distilled water and PBS buffer is observed with DLS. The crystal structure thereof is determined with XRD. The change in absorption spectrum and fluorescence emission spectrum of a quantum dot before and after modification can be detected with visible spectrophotometer. The fluorescence spectrophotometer detects fluorescence emission spectrum of quantum dots with different emission wavelengths and the fluorescence spectrum after their filling into microspheres, the spectrum change (such as change in half-peak width, red shift, blue shift and fluorescence intensity) is compared. The constant half-peak width before and after quantum dots filling has suggested that there is no aggregation among QDs. With study on microsphere fluorescence spectrum, it can be further confirm that there is no fluorescence resonance energy transfer (FRET) among multicolor QDs, and it can be solved by increasing the diameter of synthesized quantum dots if FRET occurs. Because it is required in FRET that the distance between a receptor and a donor is <10 nm, increasing the diameter of quantum dots can effectively prevent FRET among different quantum dots.

(3). Biological Coupling of Quantum Dot and Double Probes

In order to achieve the biological coupling of CdSe/ZnS quantum dot and bridged DNA and PNA, the oil-soluble CdSe/ZnS quantum dot needs to be converted into water-soluble forms. The method thereof comprises: adding 2-mercaptopropionic acid to 2 ml of oil-soluble quantum dot in toluene under stirring to react for 12 h, after 20000 rpm for 30 min, the supernatant is removed and the precipitate is washed with toluene three times followed by centrifugation, then dialysis is performed with 3.5 kD filter membrane for 12 h, carboxylated CdSe/ZnS (CdSe/ZnS—COOH) is obtained after drying and dissolved in 1×PBS (pH7.4) for storage. The fluorescence performance thereof can be detected with visible spectrophotometer. Then 100 mmol 5′ amino end modified bridged DNA probe and equimolar of 5′ amino end modified bridged PNA species-specific probe (P2) is added to 2 mmol CdSe/ZnS—COOH, the condensation reaction id performed in the present of EDC and NHS. After reaction, followed by 20000 rpm for 30 min, the supernatant is removed and the precipitate is washed with toluene three times to obtain bridged DNA labelled CdSe/ZnS quantum dot. The change in the fluorescence performance before and after DNA coupling can be detected with visible spectrophotometer and agarose gel electrophoresis is used to detect whether the coupling is successful.

(4). Study on the Change in the Fluorescence Spectrum Before and after Labelling a Probe with Quantum Dot:

The fluorescence emission spectrum and visible absorption spectrum of CdSe/ZnS quantum dot is detected with fluorescence spectrophotometer and double beam UV visible spectrophotometer respectively before and after labelling a probe with quantum dot, the extent of blue shift and red shift after labelling a probe with quantum dot is observed. Quantum dots with different colors are further ontained by changing the fluorescence wavelength of CdSe/ZnS quantum dot, and the quantum dots are coupled with DNA probes having different lengths, the fluorescence emission spectrum and visible absorption spectrum thereof is detected respectively. The relationship between the fluorescence emission spectrum, the wavelength of quantum dot and probe length before and after labelling a probe with quantum dot is established.

(5). Synthesis Characterization of Superparamagnetic Nanoparticle and its Biological Coupling with Probe

Superparamagnetic Fe₃O₄ is synthesized by chemical co-precipitation. The method thereof is as follows: mixing 0.005 mol FeCl₃ and 0.0025 mol FeSO₄ in 50 ml of double distilled water and keeping Fe³⁺/Fe²⁺=2. Then 1.5M NaOH solution is added rapidly, then the precipitate is separated after stirring for 10 minutes and washed for 4 times. Then the precipitate is washed with oxygen-free absolute ethanol and dried at 50° C. to obtain Fe₃O₄ crystal. Fe₃O₄ coated with silicon dioxide is formed by TEOS hydrolysis on the surface of Fe₃O₄. The main steps thereof is as follows: dissolving Fe₃O₄ in 240 ml ethanol, pH is adjusted to 9, 4 ml TEOS is added to react for 10 h, then heated to 50° C. to again react for 12 h. After wash with oxygen-free absolute ethanol, drying at 50° C. overnight is followed. Then Fe₃O₄ coated with silicon dioxide on surface undergoes ultrasonic dispersion in 120 ml DMF and 80 ml toluene, 10 ml APTES is added to react for 24 h, the precipitate is collected by centrifugation and washed for 3 times to obtain SiO₂@Fe₃O₄ nanoparticle with amino-modified surface. The amino-modified SiO₂@Fe₃O₄ is again dissolved in 200 ml toluene and heated to 110° C., 4.85 g glutaric anhydride is added to react for 2 h, the precipitate is collected by centrifugation and washed for 3 times to obtain SiO₂@Fe₃O₄ nanoparticle with carboxyl-modified surface (SiO₂@Fe₃O₄—COOH).

The probe labelling of superparamagnetic nanoparticle is performed with condensation of amino and carboxyl group. The method thereof comprises: 100 mmol SiO₂@Fe₃O₄—COOH is dissolved in MES (pH=5.4) buffer, then 500 mmol 5′ end amino-labelled species-specific probe (P2) is added, EDC and NHS is then added and reacted in the system for 1 h to form SiO₂@Fe₃O₄-PNA complex. The precipitate undergoes magnetic enrichment and separation followed by 4 washes, and the final precipitate is dissolved in 1×PBS buffer for storage.

(6). Performance Study on Quantum Dot Labelled Probe

Bioactivity study on quantum dot labelled probe: bioactivity is an important indicator to determine probe quality. Several oligonucleotides with different lengths (10 bp, 20 bp, 30 bp, 40 bp, 50 bp, 60 bp) are synthesized in the study and labelled with quantum dots with different colors (PNA is substituted with DNA for condition optimization to reduce experimental cost, because there is positive correlation between the different length of DNA-DNA hybridization or PNA-DNA hybridization and fluorescence intensity), then the oligonucleotides hybridize to the nucleic acid sequences that completely matches in base in DNA hybridization instrument, the hybridization efficiency is determined by the change in fluorescence intensity before and after hybridization and the probe design is optimized thereby.

Study on the retention time of quantum dot probe: PNA probes and completely matched nucleic acid sequences (DNA) are designed as above. After labelling the probes with multicolor quantum dot microspheres, the probes is stored at −20° C. away from light and taken out at 1 d, 5 d, 10 d, 20 d, 30 d, 60 d, 90 d respectively for fluorescence intensity assay with fluorescence spectrophotometer and the degradation of probe with probe hybridization assay to optimize the retention time of probe.

(7). Preparation of Nucleic Acid Sample

The short strand DNA oligonucleotide samples (<80 bp) required in methodological evaluation are synthesized by Invitrogen or Sangon Biotech. Long strand target molecule sequences are HBV DNA extracted from HBV patients diagnosed as all positive in serological examination for HBsAg, HBcAb, HBeAb. The DNA is extracted by alkaline lysis, then the extracted product is used as template and PCR amplified with designed primer pair (the primer pair is designed so that the amplified product contains the complementary sequences of P1 and P2 probes). PCR is performed again for the PCR product after gel recovery to increase purity, and the amplified product is sent to Invitrogen for sequencing. After the product to be sequenced is confirmed to contain the complementary sequences of P1 and P2 probes, such product can be used as target molecule to be tested for a methodological evaluation assay.

The verification of clinical samples requires serum from 50 health cases and 100 cases of diagnosed HBV patients (wherein cases of each subtypes are preferably collected, the invention uses HBV B/C/D genotypes as a representative because these three subtypes are the majority in our country). After intravenous collection, the whole bold sample is centrifugated at 4000 rpm for 20 min and the supernatant is collected. Nucleic acid is extracted from the collected serum by alkaline lysis and stored in an RNase-free EP tube at −80° C. for use.

(8). Study on Amplification System of Quantum Dot Signal

In order to the effectiveness of amplification system, we design a target sequence [P1-(T)₆-P2] and the both ends thereof can be completely complementary with species-specific probe P1 and P2, and we couple the two sequences with a (T)₆ linker. First prepared quantum dots labelled with amplification DNA probe (Pa) and P1 DNA probe, then the sequence complementary with amplification DNA probe (Pac) is added which contains a biotin label in end, P2 coupled magnetic beads are added in hybridization liquid to react for 30 min. Then excess streptavidin (Sa) is added, unbound chemical molecules and DNA sequences are removed from the solution with magnetic enrichment technology after complete reaction. PBS (pH7.4) buffer is again added to dissolve the precipitate (complex of magnetic bead-DNA-QD), then quantum dot with surface biotin modification (CdSe/ZnS-biotin) is added, the self-assembly of first layer of quantum dot is formed by the highly specific Sa-biotin binding. Unbound quantum dots are again removed by magnetic enrichment, the precipitate is again dissolved in PBS (pH7.4), excess Sa is added to react for 10 min, the precipitate is again dissolved in PBS after magnetic enrichment, CdSe/ZnS-biotin is added for the second time to form the self-assembly of second layer of quantum dot and so forth, the layer by layer self-assembly of quantum dot can be formed and thereby increasing a single signal to 10⁸⁻⁹ times.

Theoretically, the amplification efficiency can be calculated according to the following equation:

$$S = A {\sum }_{i = 1}^{N} 3 m \cdot {[ 3 ( n - 1 ) ]}^{𝓁 - 1} = A [ 3 m + 3 m \cdot {[ 3 ( n - 1 ) ]}^{- 1} + 3 m \cdot {[ 3 ( n - 1 ) ]}^{2} + … + 3 m \cdot {[ 3 ( n - 1 ) ]}^{N - 1} \}$$

wherein A is the copy number of DNA in the solution, m is the ssDNA number bound on every QD surface, n is the number of biotin coupled on QD surface, N is the number of layers of LBL-SA quantum dot.

With the above methods, we study the amplification factor and number of self-assemble layers of QD. The method thereof comprises: diluting the synthesized P1-(T)₆-P2 sequences to 10¹⁰ times (final concentration of 0.01 fM), then 10 ml target molecule solution is taken, 10 ul Fe₃O₄-P2 and 10 ul 540 nm QD-P1 solution is added to PBS (pH7.4) buffer to hybridize for 20 min, then an external magnetic field of 0.3 T is applied for 3 min, the hybridized target molecule-magnetic bead-quantum dot complex is separated and washed with PBS (pH7.4) buffer for 3 times respectively, then the complex is again dissolved in 1 ml PBS (pH7.4), at the same time 100 μl 1 mM streptavidin is added to react for 10 min, then an external magnetic field of 0.3 T is applied for separation, the complex is washed for 3 times and dissolved in 1 ml PBS (pH7.4), then 100 μl 1 mM biotin-labelled 540 nmQD is added to react for 10 min, the self-assembly of the first layer of QD is formed after separation with an external magnetic field and wash. At this time, the fluorescence intensity of the complex is recorded as FL1. 100 μl 1 mM streptavidin and 100 μl 1 mM biotin-labelled 540 nmQD are successively added as the above method, the self-assembly of the second layer of QD is obtained after magnetic enrichment and separation, the fluorescence intensity of the complex is recorded as FL2. According to the same method, the fluorescence intensity of the self-assembly of the third, fourth . . . layer of QD is recorded as FL3, FL4, FL5 . . . until FL10 from the self-assembly of the tenth layer of QD. The result is shown as figures, the self-assembly of the first layer of QD can amplify a signal to 12 times, the second layer can amplify a signal to 174 times, the third can amplify a signal to 1634 times and so forth to 1.13E8 times of the original fluorescence intensity in the tenth layer. Therefore, our practical detection result is close to but a little lower than the theoretical result, the cause thereof may be the space steric effect after multiple layer amplification, which leads to incomplete assembly during the multiple layer assembly of quantum dot.

(9). Simultaneous Detection and Genotyping

When the quantum dot labelled P1 probe and magnetic microsphere labelled P2 probe is added to the solution containing a target molecule (T), the hybridization is performed for 30 min which is followed by magnetic separation, the resulted precipitate is a complex containing 540 nm QD-P1, Fe₃O₄-P2 and the target molecule (P1-T-P2). As P1 and P2 are species-specific probes against different sites, the complex detect all HBV viral DNA. 620 nm CdSe/ZnS labelled genotyping probe (P3) is further added. As P3 can complementarily hybridize to the specific site in the target molecule, different genotypes can be determined by different colors. The target molecule complex to be detected (P1-P2-P3-T) can be separated from the system by magnetic separation and the detection is performed with fluorescence spectral imaging technology or flow cytometry. As P2 and P3 are labelled with different colors, the detection and simultaneous genotyping is performed with the finally detected color. Furthermore, the co-occurrence of the two colors can be used as self-reference and the occurrence of only the color of P3 probe (no color of P2 probe) is false positive. Likewise, the occurrence of only the color of P2 probe (no color of P3 probe) illustrates false positive result produced during the amplification of P2 probe signal. True positive results can only be confirmed by the co-occurrence of the colors of P2 and P3 and thereby increasing the specificity of detection. In this example, we design the following corresponding probes for different genotypes and said probes are labelled with quantum dot: B genotype probe (P3b-QD560 nm), C genotype probe (P3c-QD580 nm) and D genotype probe (P3d-QD620 nm), typing probes for different genotypes can be established with the same method. The result shows that P3b-QD620 nm can be separated from 540 nm identification probe without overlapping in light spectrum, therefore identification can be performed accurately. The signal is weak because the identification probe is not amplified at this time, if amplified, the signal thereof can reach the fluorescence intensity as 540 nm QD.

The PNA sequences of various typing probes are:

| Probe name | Sequence |
|---|---|
| P3b | 5′-NH₂-(CH2)₆-TGTGTTTACTGAGTG-3′ |
| P3c | 5′-NH₂-(CH2)₆-AACGCCCACATGATCT-3′ |
| P3d | 5′-NH₂-(CH2)₆-CGGTACGAGATCTTCTA-3′ |

It should be understood that these embodiments are merely illustrative of the invention and are not intended to limit the scope of the invention. Those skilled in the art can make various modifications or improvement to the present invention, and these equivalent forms also fall within the present application as defined by the appended claims scope.

1. A method for detecting and typing nucleic acids of pathogenic microorganism without amplification, wherein the method comprises the following steps:
(1) Synthesizing DNA and/or PNA probe 1, 2, 3 according to the nucleic acid sequence of a sample to be tested, the probe 1, 2, 3 can hybridize to the sample to be tested respectively without overlapping with each other;
(2) Coupling the probe 1, 2, 3 with a magnetic nanoparticle and two fluorescence quantum dots respectively, the fluorescence of the fluorescence quantum dots can be same or different;
(3) Synthesizing biotin-linked bridged DNA and/or PNA sequences 1, 2 and complementary sequences 1′, 2′, the sequences 1′ and 2′ are coupled with the two fluorescence quantum dots in step (2) respectively;
(4) Synthesizing two biotin-modified fluorescence quantum dots with different fluorescence colors, the two fluorescence quantum dots and the fluorescence quantum dots in step (2) can be same or different;
(5) Selecting the probe-modified magnetic nanoparticle and one of the probe-modified fluorescence quantum dots in step (2), which hybridize to the sample to be tested and corresponding bridged sequences, followed by magnetic separation; then layer by layer assembly is performed by repeating as follows: adding Sa (streptavidin)-wash-adding one of the biotin-modified fluorescence quantum dots in step (4)-wash, then the concentrate of the sample to be tested is obtained by magnetic separation, optionally a sample of the concentrate is measured for fluorescence intensity;
(6) Selecting the other probe-modified fluorescence quantum dot, which then hybridizes to the concentrate from step (5) and the corresponding bridged sequence, followed by magnetic separation; then layer by layer assembly is performed by repeating as follows: adding Sa-wash-adding one of the biotin-modified fluorescence quantum dots in step (4)-wash, then the second concentrate of the sample to be tested is obtained by magnetic separation, optionally a sample of the second concentrate is measured by fluorescence spectral imaging technology or flow cytometry.

2. A kit for detecting and typing nucleic acids of pathogenic microorganism without amplification, wherein the kit comprises: three DNA and/or PNA probe-coupled magnetic nanoparticles and two fluorescence quantum dots, the three DNA and/or PNA probe-coupled magnetic nanoparticles can hybridize to a sample to be tested without overlapping with each other, the fluorescence of the fluorescence quantum dots can be same or different; biotin-modified bridged DNA and/or PNA, two fluorescence quantum dots coupled with the complementary sequence of bridged DNA and/or RNA; two biotin-modified fluorescence quantum dots with different fluorescence colors, and the fluorescence of the two fluorescence quantum dots and the above fluorescence quantum dots can be same or different; SA and a buffer.

3. The method according to claim 1, wherein the sample to be tested is HBV nucleic acid.

4. The method according to claim 1, wherein the probe is PNA, one or more or all of the fluorescence quantum dots is CdSe/ZnS quantum dot, preferably, the emission wavelengths of two different quantum dots differ by at least 30 nm, preferably at least 50 nm, more preferably at least 80 nm.

5. The method according to claim 1, wherein the magnetic nanoparticle is SiO₂@Fe₃O₄ nanoparticle.

6. The method according to claim 1, wherein the three probes are PNA, wherein two species-specific probe sequences is the sequences of PNA species-specific probe 1 and/or PNA species-specific probe 2:

| Probe name | Probe sequence |
|---|---|
| PNA species- | 5′-NH₂-(CH2)₆-AGGCACAGCTTGGAG |
| specific probe 1 | GC-3′ |
| PNA species- | 5′-NH₂-(CH2)₆-GTGATGTGCTGGGTG |
| specific probe 2 | TGTCG-3′ |

7. The method according to claim 6, wherein the other sequence for typing is selected from one of the following three probes:

| Probe name | Sequence |
|---|---|
| P3b | 5′-NH₂-(CH2)₆-TGTGTTTACTGAGTG-3′ |
| P3c | 5′-NH₂-(CH2)₆-AACGCCCACATGATCT-3′ |
| P3d | 5′-NH₂-(CH2)₆-CGGTACGAGATCTTCTA-3′ |

8. The method according to claim 7, wherein the sequence of the bridged DNA is 5′-biotin-GGGCAGCTGGGGCGGGCGGG-NH₂-3′.

9. The method according to claim 1 wherein the method is not for diagnose.

10. The kit according to claim 2, wherein the probe is PNA, one or more or all of the fluorescence quantum dots is CdSe/ZnS quantum dot, preferably, the emission wavelengths of two different quantum dots differ by at least 30 nm, preferably at least 50 nm, more preferably at least 80 nm.

11. The kit according to claim 2, wherein the magnetic nanoparticle is SiO₂@Fe₃O₄ nanoparticle.

12. The kit according to claim 2, wherein the three probes are PNA, wherein two species-specific probe sequences is the sequences of PNA species-specific probe 1 and/or PNA species-specific probe 2:

| Probe name | Probe sequence |
|---|---|
| PNA species- | 5′-NH₂-(CH2)₆-AGGCACAGCTTGGAGG |
| specific probe 1 | C-3′ |
| PNA species- | 5′-NH₂-(CH2)₆-GTGATGTGCTGGGTGT |
| specific probe 2 | GTCG-3′ |

13. The kit according to claim 12, wherein the other sequence for typing is selected from one of the following three probes:

| Probe name | Sequence |
|---|---|
| P3b | 5′-NH₂-(CH2)₆-TGTGTTTACTGAGTG-3′ |
| P3c | 5′-NH₂-(CH2)₆-AACGCCCACATGATCT-3′ |
| P3d | 5′-NH₂-(CH2)₆-CGGTACGAGATCTTCTA-3′ |

14. The kit according to claim 13, wherein the sequence of the bridged DNA is 5′-biotin-GGGCAGCTGGGGCGGGCGGG-NH₂-3′.

15. The kit according to claim 2 wherein the method is not for diagnose.


SEQUENCE LISTING (6 sequences)

SEQ ID NO: 1 - DNA, 17 bp, Artificial Sequence
  Artificially synthesized
aggcacagcttggaggc

SEQ ID NO: 2 - DNA, 20 bp, Artificial Sequence
  ARTIFICIALLY SYNTHESIZED
gtgatgtgctgggtgtgtcg

SEQ ID NO: 3 - DNA, 20 bp, Artificial Sequence
  ARTIFICIALLY SYNTHESIZED
gggcagctggggcgggcggg

SEQ ID NO: 4 - DNA, 15 bp, Artificial Sequence
  ARTIFICIALLY SYNTHESIZED
tgtgtttactgagtg

SEQ ID NO: 5 - DNA, 16 bp, Artificial Sequence
  ARTIFICIALLY SYNTHESIZED
aacgcccacatgatct

SEQ ID NO: 6 - DNA, 17 bp, Artificial Sequence
  ARTIFICIALLY SYNTHESIZED
cggtacgagatcttcta